(19) United States
(12) Patent Application Publication
(10) Pub. No.: US 2013/0158476 A1
(43) Pub. Date: Jun. 20, 2013

(54) SYSTEM AND METHOD FOR SYNCHRONIZING PHYSICAL AND VISUALIZED MOVEMENTS OF A MEDICAL DEVICE AND VIEWING ANGLES AMONG IMAGING SYSTEMS

(71) Applicant: Eric S. Olson, Maplewood, MN (US)

(72) Inventor: Eric S. Olson, Maplewood, MN (US)

(21) Appl. No.: 13/326,600

(22) Filed: Dec. 15, 2011

(51) Int. Cl. A61M 25/092

(52) U.S. Cl. 604/95.01

(57) ABSTRACT

A system and method for synchronizing movement of a device within a body with a desired movement of the device commanded through a user interface are provided: The system includes an electronic control unit (ECU) configured to determine a viewing angle of an imaging system that captures an image of the device within the body and generates the image on a display. The ECU is configured to receive a command through the user interface, the command indicative of the desired movement of the device on the display and to generate a control signal to control movement of the device within the body responsive to the command and the viewing angle. The ECU may further be configured to generate a model of a region of interest and illustrate the position of the device and to adjust a display angle of the model responsive to the viewing angle of the imaging system.

### BACKGROUND OF THE INVENTION

a. Field of the Invention

This invention relates to a system and method for synchronizing movement of a medical device within a body with a desired movement of the device commanded through a user interface. In particular, the instant invention relates to a system and method in which intuitive control of the device is provided such that commanded movements of the device relative to a visual display of the device are translated into corresponding physical movements of the device.

b. Background Art

A wide variety of medical devices are inserted into the body to diagnose and treat a various medical conditions. Catheters, for example, are used for to perform a variety of tasks within human bodies and other bodies including the delivery of medicine and fluids, the removal of bodily fluids and the transport of surgical tools and instruments. In the diagnosis and treatment of atrial fibrillation, for example, catheters may be used to deliver electrodes to the heart for electrophysiological mapping of the surface of the heart and to deliver ablative energy to the surface among other tasks. Catheters are typically routed to a region of interest through the body's vascular system. The catheter may be advanced and retracted manually by the clinician. Manual movement of the catheter requires precise control and is dependent on the skill of the clinician. In order to reduce or eliminate potential variability in the procedure due to clinician skill and to allow performance of procedures from remote locations, remote catheter guidance systems (RCGS) have been developed using electromechanical drive systems to control catheter movement. Several embodiments of an RCGS are disclosed and illustrated in U.S. Published Patent Application No. 2010-0256558, U.S. Pat. No. 6,507,751 and U.S. Published Patent Application No. 2007-0016006, the entire disclosures of which are incorporated herein by reference.

An RCGS can be designed to allow intuitive control of the catheter by the clinician. A conventional navigation system such as the system sold under the trademark “ENSITE NAVX” by St. Jude Medical, Inc. can be used to track the position of the catheter and display the catheter position relative to a three-dimensional model of a region of interest such as the heart. A clinician can use a user interface to direct movement of the catheter relative to the model as shown in the display. The physical relationship of the clinician relative to the model as shown on the display screen, however, may not match the physical relationship of the clinician relative to the patient's body. As a result, a commanded movement of the catheter in one direction relative to the model in the display could result in a movement of the actual catheter in the body in a different direction relative to the region of interest in the body if the commanded movements were directly transferred to the catheter. As disclosed in U.S. Published Application No. 2010/0256558, an RCGS can be designed to perform translation and rotation of the command movements input at the user interface to impart corresponding movements to the actual catheter such that a commanded movement of the catheter in one direction relative to the model in the display results in the same movement of the catheter relative to the region of interest in the body.

Providing intuitive control of catheter movements relative to movements of a catheter on a three-dimensional model of the type generated in conventional catheter navigation systems has many benefits. The position of the catheter relative to the model as shown on the display, however, may not precisely match the position of the catheter relative to the region of interest in the body due to the passage of time between measurement and display of catheter position. Further, the possibility exists that the navigation system could be rendered inoperable thereby leaving the clinician without the ability to accurately control further movement of the catheter.

The inventor herein has recognized a need for a system and method for synchronizing movement of a device within a body with a desired movement of said device commanded through a user interface that will minimize and/or eliminate one or more of the above-identified deficiencies.

### BRIEF SUMMARY OF THE INVENTION

It is desirable to provide a system and method for synchronizing movement of a device within a body with a desired movement of said device commanded through a user interface. In particular, it is desirable to provide a system and method that will enable real time intuitive control of device movements within the body.

A system for synchronizing movement of a device within a body with a desired movement of the device commanded through a user interface in accordance with one embodiment of the invention includes an electronic control unit configured to determine a viewing angle of an imaging system configured to capture an image of the device within the body and to generate the image on a first display. The imaging system may, for example, comprise a fluoroscopic imaging system. The electronic control unit is further configured to receive a command through the user interface, the command indicative of the desired movement of the device on the first display. The electronic control unit is further configured to generate a control signal to control movement of the device within the body responsive to the command and the viewing angle of the imaging system. In accordance with another embodiment of the invention, the electronic control unit may be further configured to determine a position of the device within the body and generate an image on a second display, the image including a model of a region of interest in the body and a representation of the device located relative to the model based on the position. The electronic control unit may be further configured to adjust a display angle of the model responsive to the viewing angle of the imaging system.

A method for synchronizing movement of a device within a body with a desired movement of said device commanded through a user interface in accordance with one embodiment of the invention includes the step of determining a viewing angle of an imaging system configured to capture an image of the device within the body and to generate the image on a first display. The method further includes the step of receiving a command through the user interface, the command indicative of the desired movement of the device on the first display. The method further includes the step of generating a control signal to control movement of the device responsive to the command and the viewing angle. In accordance with another embodiment of the invention, the method may further include the steps of determining a position of the device within the body and generating an image on a second display, the image including a model of a region of interest in the body and a representation of the device located relative to the model based on the position. The method may further include the step of adjusting a display angle of the model responsive to the viewing angle of the imaging system.

A system and method in accordance with the present invention are advantageous because the system and method provide real time intuitive control of the device. In particular, imaging systems such as a fluoroscopic imaging system provide a substantially real time display of the position of the device within the body thereby eliminating any lag in time between actual and displayed movements. The inventive system and method also provide a safeguard against the possibility that the navigation system could be rendered inoperable and leave the clinician without the ability to accurately control further movement of the device.

The foregoing and other aspects, features, details, utilities and advantages of the present invention will be apparent from reading the following description and claims, and from reviewing the accompanying drawings.

### BRIEF DESCRIPTION OF THE DRAWINGS

FIG. 1 is a diagrammatic view of a system for synchronizing movement of a device within a body with a desired movement of the device commanded through a user interface in accordance with one embodiment of the present invention.

FIG. 2 is a diagrammatic view of a remote catheter guidance system illustrating an exemplary layout of various system components.

FIG. 3 is a diagrammatic view of one component of the remote catheter guidance system of FIG. 2.

FIG. 4 is a flow chart diagram of a method for synchronizing movement of a device within a body with a desired movement of the device commanded through a user interface in accordance with one embodiment of the present invention

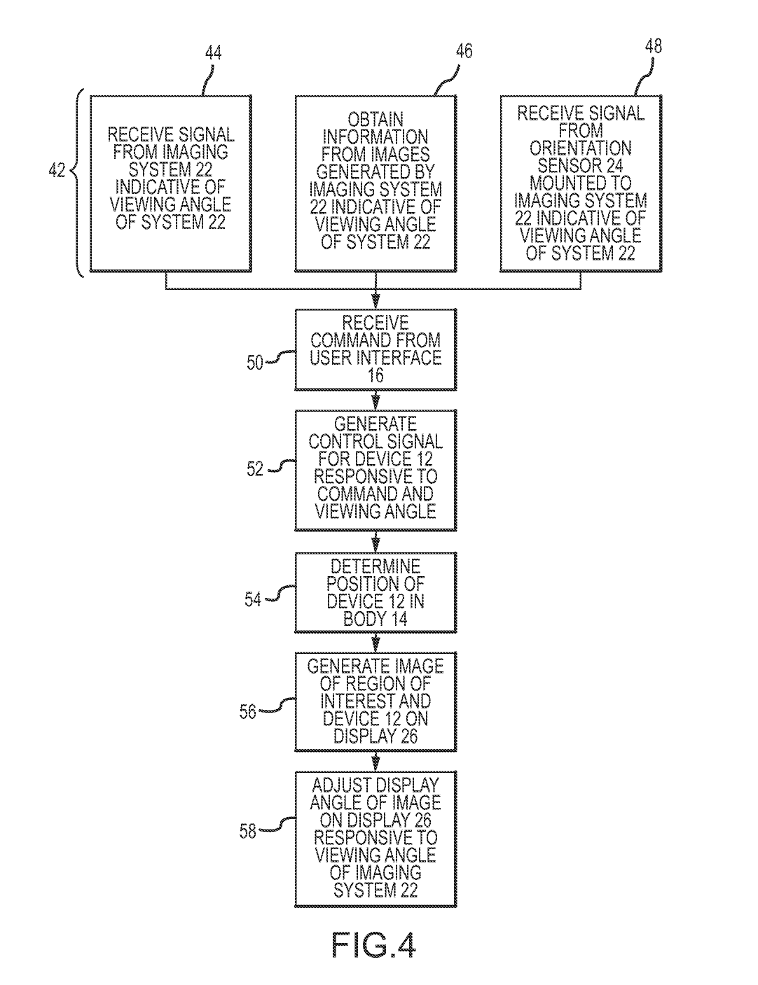

### DETAILED DESCRIPTION OF EMBODIMENTS OF THE INVENTION

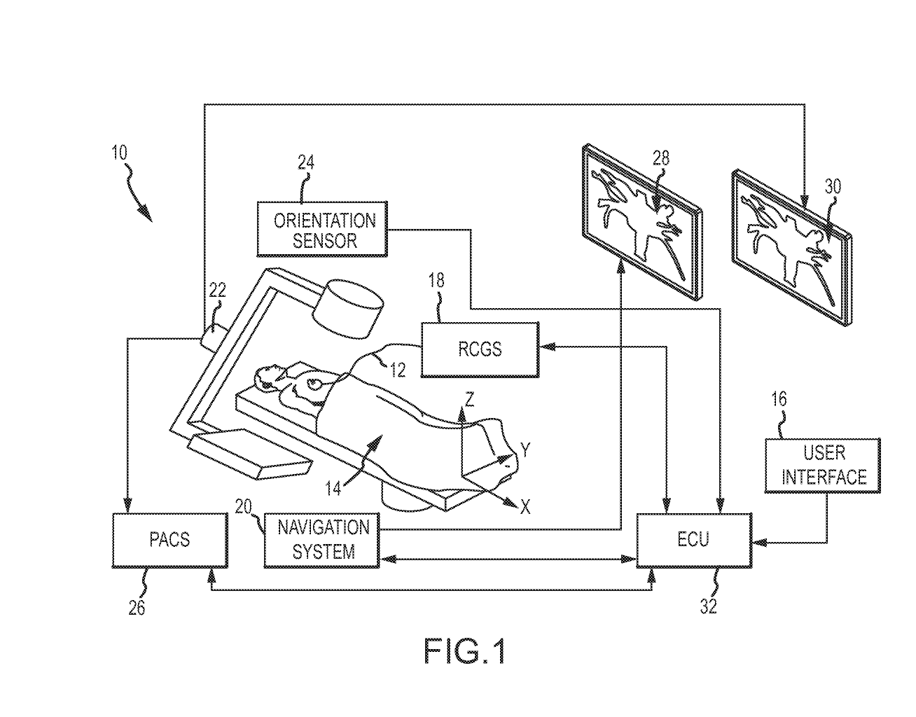

Referring now to the drawings wherein like reference numerals are used to identify identical components in the various views, FIG. 1 illustrates a system 10 for synchronizing movement of a device 12 within a body 14 with a desired movement of the device commanded through a user interface 16 in accordance with one embodiment of the invention. Device 12 is provided for use in diagnostic or treatment procedures on body 14. Device 12 may comprise a catheter for delivery of medicine or fluids to a region of interest in body 14, removal of bodily fluids and/or transporting surgical tools or instruments within body 14. Device 12 may comprise, for example, an electrophysiological (EP) catheter (contact or non-contact) for use in gathering EP data associated with cardiac tissue. Device 12 may alternatively comprise an intracardiac electrocardiography (ICE) catheter for generating an internal image of the heart such as one of the catheters sold by St. Jude Medical, Atrial Fibrillation Division, Inc. under the registered trademark “VIEWFLEX.” Device 12 may alternatively comprise an ablation catheter for providing ablation energy (e.g., radiofrequency, ultrasound, cryogenic, laser or other light) to tissue, such as cardiac tissue, within body 14. Although a catheter has been used herein as specific example of device 12, it should be understood that device 12 may comprise a variety of conventional diagnostic and treatment devices other than a catheter including, for example, an introducer sheath. In addition to user interface 16, system 10 may include a remote catheter guidance system (RCGS) 18, a navigation system 20, an imaging system 22, an orientation sensor 24, a picture archiving and communication system (PACS) 26, displays 28, 30, and one or more electronic control units (ECU) 32.

User interface 16 is provided for a clinician to control device 12 within body 14. In particular, interface 16 allows the clinician to interact with the RCGS 18 to control movement of device 12. For example, in the case of a catheter, interface 16 enables the clinician to control advancement and retraction of the catheter and deflection of the catheter tip. Interface 16 may assume a variety of conventional forms including various two-dimensional and three-dimensional input devices such as a mouse, joystick, instrumented user-wearable gloves, touch screen display monitors, and spatially detected styluses. Where device 12 is a catheter, interface 16 may comprise traditional catheter handle controls or oversized catheter models. Interface 16 may be configured to directly control the movement of device 12, or may be configured, for example, to manipulate a target or cursor on an associated display 28, 30. Potential embodiments of interface 16 and various features of interface 16 described in greater detail in U.S. Published Application No. 2010-0256558, the entire disclosure of which is incorporated herein by reference.

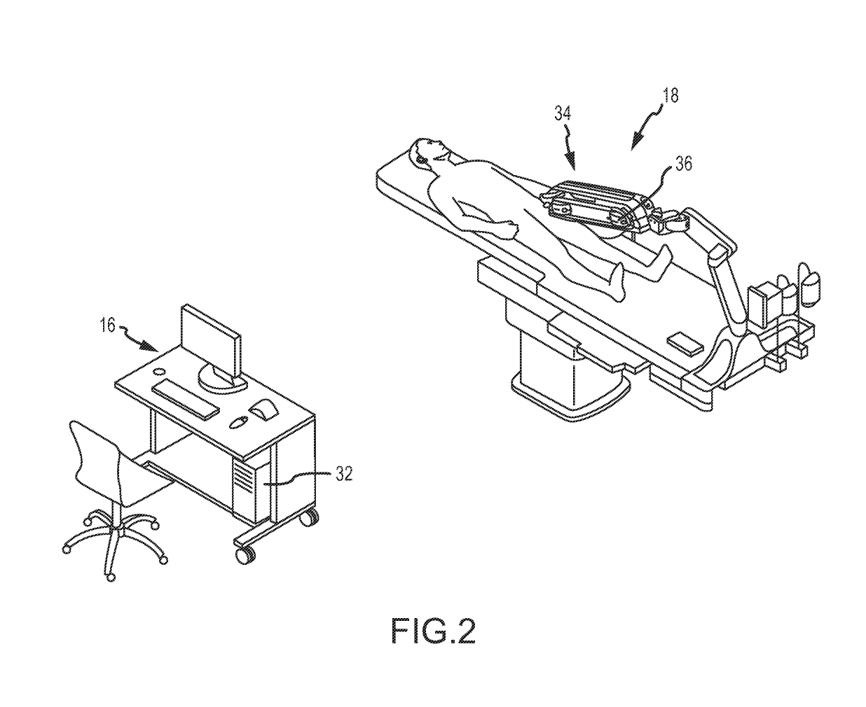

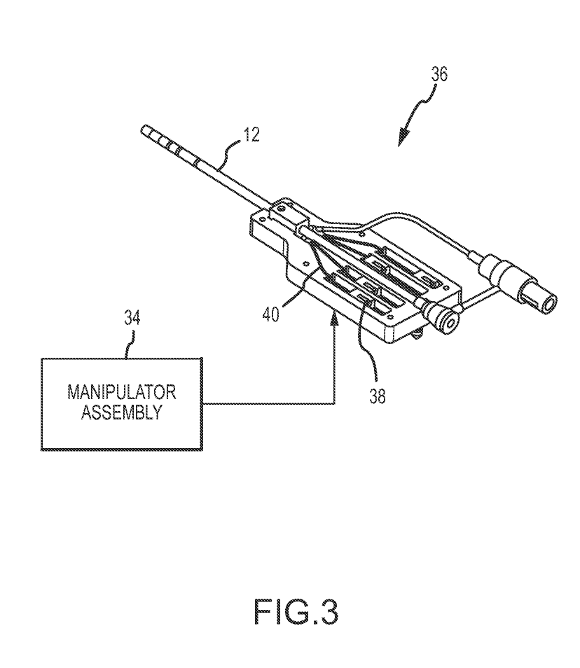

RCGS 18 is provided to manipulate device 12. In the case of a catheter, RCGS 18 permits control of translation, distal bending, and virtual rotation of the catheter and any surrounding sheath. RCGS 18 therefore provides the user with a type of control similar to that provided by conventional manually-operated systems, but allows for repeatable, precise, and dynamic movements. A clinician may identify desired movements of device 12 and/or target locations (potentially forming a path) on an image. RCGS 18 relates these movements and digitally selected points to positions within the patient's actual/physical anatomy, and may thereafter control the movement of device 12 to defined positions where the clinician or the RCGS 18 can perform the desired diagnostic of therapeutic function. Referring to FIGS. 2-3, RCGS 18 may include a manipulator assembly 34 for operating a device cartridge 36. In addition, user interface 16 and ECU 32 may be considered a part of RCGS 18 with ECU 32 configured to translate (i.e., interpret) inputs (e.g., motions) of the user at user interface 16 into a resulting movement of device 12. ECU 32 issues commands to manipulator assembly 34 (i.e., to the actuation units—electric motors) to move or bend device 12 to prescribed positions and/or in prescribed ways, all in accordance with the received user input and a predetermined programmed operating strategy.

Manipulator assembly 34 is configured to maneuver device 12 in response to commands from ECU 32. In the case of a catheter, assembly 34 may cause translational movement such as advancement or withdrawal of the catheter and effect deflection of distal end of the catheter and/or rotation or virtual motion. Assembly 34 may include conventional actuation mechanisms (e.g., a plurality of electric motor and lead screw combinations) for linearly actuating one or more control members (e.g., steering wires) associated with the catheter for achieving the above-described translation, deflection, and/or rotation (or virtual rotation).

Device cartridge 36 is provided to translate movement of elements in manipulator assembly 34 to device 12. Cartridge 36 receives and retains the proximal end of device 12. In the case of a catheter, cartridge 36 may include sliding blocks 38 each coupled to a corresponding steering wire 40 so as to permit independent tensioning of each wire 40. Movement of the blocks 38 is controlled by manipulator assembly 34 to cause tensioning of the wires 40 and thereby affect translation, deflection, and rotation of device 12.

A more complete description of various embodiments of an RCGS may be found in the following patent applications that are incorporated herein by reference: U.S. Published Patent Application No. 2011-0015569 filed Sep. 16, 2010 and titled “Robotic Catheter System Input Device”; U.S. Published Patent Application No. 2010-0256558 filed Mar. 31, 2010 and titled “Robotic Catheter System”; U.S. Published Patent Application No. 2009-0247944 filed Dec. 13, 2008 and titled “Robotic Catheter Rotatable Device Cartridge”; U.S. Published Patent Application No. 2009-0247942 filed Dec. 31, 2008 and titled “Robotic Catheter Manipulator Assembly”; U.S. Published Patent Application No. 2009-0247993 filed Dec. 31, 2008 and titled “Robotic Catheter System”; U.S. Published Patent Application No. 2009-0248042 filed Dec. 31, 2008 and titled “Model Catheter Input Device” and International Patent Application No. PCT/US2009/038597 filed Mar. 29, 2009 and titled “Robotic Catheter System With Dynamic Response” (published as WO 2009/120982).

Navigation system 20 is provided to determine the position and orientation of device 12 within body 14 and may also be used to generate an electrophysiological map of a region of interest. System 20 may display geometries or models of a region of interest in body 14 on a display such as display 28 along with a representation of device 12 indicative of the position of device 12 relative to the region of interest. System 20 may also display activation timing and voltage data for cardiac tissue on the same display 28. System 20 may, for example, comprise the system offered for sale under the trademark “ENSITE NAVX” by St. Jude Medical, Inc. and described in U.S. Pat. No. 7,263,397 titled “Method and Apparatus for Catheter Navigation and Location Mapping in the Heart,” the entire disclosure of which is incorporated herein by reference. The system is based on the principle that when low amplitude electrical signals are passed through the thorax, body 14 acts as a voltage divider (or potentiometer or rheostat) such that the electrical potential or field strength measured at an electrode on device 12 may be used to determine the position of the electrode, and therefore device 12, relative to a pair of external patch electrodes using Ohm's law and the relative location of a reference electrode (e.g. in the coronary sinus). In one configuration, the system includes three pairs of patch electrodes that are placed on opposed surfaces of body 14 (e.g., chest and back, left and right sides of the thorax, and neck and leg) and form generally orthogonal x, y, and z axes as well as a reference electrode/patch that is typically placed near the stomach and provides a reference value and acts as the origin of the coordinate system for the navigation system 20. Sinusoidal currents are driven through each pair of patch electrodes and voltage measurements for one or more position sensors (e.g., electrodes) associated with device 12 are obtained. The measured voltages are a function of the distance of the position sensors from the patch electrodes. The measured voltages are compared to the potential at the reference electrode and a position of the position sensors within the coordinate system of the navigation system 20 is determined.

In an alternative embodiment, system 20 may comprise a system that employs magnetic fields to detect the position of device 12 within body 14 such as the system offered for sale under the trademark “GMPS” by MediGuide, Ltd. and generally shown and described in, for example, U.S. Pat. No. 7,386,339 entitled “Medical Imaging and Navigation System,” the entire disclosure of which is incorporated herein by reference or the system offered for sale under the trademark “CARTO XP” by Biosense Webster, Inc. and generally shown and described in, for example, U.S. Pat. No 6,690,963, the entire disclosure of which is incorporated herein by reference. In such a system, a magnetic field generator may be employed having multiple emitter coils, arranged in different locations and orientations to create magnetic fields within body 14 and to control the strength, orientation, and frequency of the fields. The magnetic field generator may be located above or below the patient (e.g., under a patient table) or in another appropriate location. Magnetic fields are generated by the emitter coils and current or voltage measurements for one or more position sensors (e.g., a sensor coil) associated with device 12 are obtained. The measured currents or voltages are proportional to the distance of the sensors from the emitter coils and a function of the relative orientation of the sensor coil to the emitter coils, thereby allowing the position and orientation of the sensors within the coordinate system of system 20 to be determined.

Imaging system 22 is provided to acquire images of the heart or another anatomical region of interest in body 14 and is conventional in the art. In the illustrated embodiment, imaging system 22 comprises a fluoroscopic imaging system. It should be understood, however, that the invention described herein may find use with other types of imaging systems including, for example, but without limitation, computed tomography (CT) imaging systems, magnetic resonance (MR) imaging systems, ultrasound imaging systems, positron emission tomography (PET) imaging systems, and single-photon emission computed tomography (SPECT) systems. Imaging system 22 captures images of a region of interest in body 14 containing device 12. System 22 may transmit images to PACS 26 for archival and distribution and/or to display 30. Images captured by system 22 may be compatible with the Digital Imaging and Communications in Medicine (DICOM) standard promulgated by the National Electrical Manufacturers Association (NEMA). Additional information regarding this file format can be found in the published standard titled “Digital Information and Communications in Medicine (DICOM) PS 3.1 2009,” National Electrical Manufacturers Association (copyright 2009), the entire disclosure of which is incorporated herein by reference. For a purpose described hereinbelow, each image may include information about the viewing angle of imaging system 22 when the image was taken. Imaging system 22 may also output this information about the viewing angle directly to ECU 32.

Orientation sensor 24 may be provided to generate an output signal indicative of the viewing angle of imaging system 22. Orientation sensor 24 may be mounted directly or indirectly on imaging system 22 and may communicate with ECU 32 over a wired (e.g. through a USB cable, Ethernet connection or fiber optic connection) or wireless (e.g., infrared, radio-frequency etc.) connection. In one embodiment of the invention, sensor 24 comprises an inclinometer which may employ three orthogonal force sensors, such as accelerometers, to measure a change in force due to gravity as imaging system 22 rotates and thereby provide an indication of the viewing angle of imaging system 22 relative to the region of interest in body 14. In an alternative embodiment of the invention, sensor 24 may comprise a gyroscope. In yet another embodiment of the invention, sensor 24 may comprise a magnetic field position sensor such as a coil responsive to magnetic fields generated by versions of system 20 employing magnetic fields for position detection. The magnetic field position sensor may be mounted on imaging system 22, on body 14 or on a table supporting body 14.

Picture archiving and communications system (PACS) 26 provides storage (“archiving”) and access (“communications”) to images from various imaging modalities. PACS 26 is conventional in the art and may comprise a conventional server configured to communicate with various electronic storage devices including devices for writing and reading to electronic storage media such as compact discs. PACS 26 may, for example, comprise a server running software sold under the trademark “HORIZON MEDICAL IMAGING” by McKesson Corp. or under the trademark “SYNGO.PLAZA” by Siemens AG. PACS 26 is typically configured to archive files in accordance with the DICOM standard.

Displays 28, 30 are provided to convey information to a clinician to assist in diagnosis and treatment. Displays 28, 30 may comprise one or more conventional computer monitors or other display devices. Displays 28, 30 may provide a graphical user interface (GUI) to the clinician. The GUI may include a variety of information including, for example, an image of the geometry of a region of interest in body 14, associated electrophysiology data, graphs illustrating voltage levels over time for various electrodes on device 12, and images of device 12 and other medical devices and related information indicative of the position of device 12 and other devices relative to the region of interest.

ECU 32 provides a means for controlling the movement of device 12 within body 14. ECU 32 is configured to translate (i.e., interpret) inputs or commands (e.g., motions) of the user at interface 16 or from another source into a resulting movement of device 12. ECU 32 issues commands to manipulator assembly 34 (i.e., to the actuation units—electric motors) to move or bend device 12 to prescribed positions and/or in prescribed ways, all in accordance with the received user input and a predetermined programmed operating strategy. ECU 32 may also be a component of navigation system 20 and thereby provide a means for determining the geometry of a region of interest in body 14, electrophysiology characteristics of the region of interest and the position and orientation of device 12 relative to the region of interest. ECU 32 may also be a component of imaging system 22. ECU 32 also provides a means for generating display signals used to control displays 28, 30. ECU 32 may comprise one or more programmable microprocessors or microcontrollers or may comprise one or more ASICs. ECU 32 may include a central processing unit (CPU) and an input/output (I/O) interface through which ECU 32 may receive a plurality of input signals including signals generated by electrodes on device 12, inputs or commands on interface 16, feedback signals from RCGS 18 and components of navigation system 20 and imaging system 22 and generate a plurality of output signals including those used to control and/or provide data to device 12, manipulator assembly 34 of RCGS 18 and displays 28, 30. Although a single ECU 32 is shown in the illustrated embodiment for use with RCGS 18, navigation system 20 and imaging system 22, it should be understood that RCGS 18, navigation system 20 and imaging system 22 may be configured with individual ECUs.

In accordance with the present teachings, ECU 32 may be configured with programming instructions from a computer program (i.e., software) to implement a method for synchronizing movement of device 12 within body 14 with a desired movement of device 12 commanded through user interface 16. Referring to FIG. 3, the method may begin with the step 42 of determining a viewing angle of an imaging system, such as imaging system 22, that is configured to capture an image of device 12 within body 14 and to generate the image on a display 26. ECU 32 may determine the viewing angle of imaging system 22 in several different ways. In one embodiment of the invention, step 42 includes substep 44 of receiving a signal generated by imaging system 22 indicative of the viewing angle of imaging system 22. Conventional imaging systems 22 may include systems for monitoring the viewing angle of the imaging system 22 and may store this data in an accessible memory or directly output this data to another device or system such as ECU 32. Because of variance in data handling and communication protocols among imaging system manufacturers, however, direct access to this data may not always be possible. Therefore, in accordance with another embodiment of the invention, step 42 includes the substep 46 of obtaining information from images generated by imaging system 22 indicative of the viewing angle of imaging system 22. DICOM images generated by imaging system 22 will include data about the viewing angle of imaging system 22 when the image was captured. ECU 32 may therefore obtain data regarding the viewing angle of system 22 from the captured images. Substep 46 may therefore include the further substep of accessing an image in PACS 26 to retrieve data about the viewing angle. Obtaining data about the viewing angle from the standard DICOM images output by imaging systems 22 overcomes the issue of variance in data handling and communications protocols among different imaging system manufacturers. Retrieving data from the images, however, introduces a time delay that may be dependent on speed of access and communication with PACS 26 which may be undesirable.

In accordance with yet another embodiment of the invention, step 42 includes the substep 48 of receiving an output signal generated by orientation sensor 24 mounted on imaging system 22. As noted above, obtaining information directly from imaging system 22 may not be possible due to variations in data handling and communications protocols among imaging system manufacturers. Retrieving data from images captured by imaging system 22 may introduce an undesirable time delay. Use of orientation sensor 24 overcomes both of these issues because it is independent of the data handling and communications protocols of the various imaging systems and does not require access to acquired images to retrieve position data. It should be noted, however, that orientation sensor 24 is appropriate for use with imaging systems, such as conventional C-arm fluoroscopic imaging systems, in which the viewing angle is determined by a rotating structure to which orientation sensor 24 may be affixed. Imaging systems lacking such a structure may employ either of the other embodiments of the invention discussed hereinabove.

ECU 32 receives output signals from orientation sensor 24 indicative of the viewing angle of imaging system 22. Referring to FIG. 1, in the case of a conventional C-arm imaging system, rotation is possible along an x-axis that extends in the longitudinal direction of the table on which the patient lays and a y-axis that extends in a lateral direction to the table on which the patient lays. Rotation does not occur along a z-axis extending vertically through the patient table, but, in the case of an inclinometer as the orientation sensor 24, the force measurement along the z-axis would remain the same anyway because the z-axis is parallel to the force of gravity. A rotation matrix corresponding to the viewing angle and consisting of three rotational matrices, R=RₓR_{y}R_{z}, about the three orthogonal axes can therefore be defined as R=RₓR_{y} where R_{z}=1. ECU 32 determines the rotation matrix R in response to information generated by orientation sensor 24. In particular, the rotation matrix has three degrees of freedom derived from the rotation angles θ, φ, γ about each of the x, y, and z axes. Because γ is constrained to be zero, the matrix Rₓ and R_{y} may be defined as:

$${R}_{X} = [ \begin{matrix}1 & 0 & 0 \\ 0 & cos ( θ ) & - sin ( θ ) \\ 0 & sin ( θ ) & cos ( θ )\end{matrix} ]$$

$$and$$

$${R}_{y} = [ \begin{matrix}cos ( ϕ ) & 0 & sin ( ϕ ) \\ 0 & 1 & 0 \\ - sin ( ϕ ) & 0 & cos ( ϕ )\end{matrix} ]$$

The bottom row of rotation matrix R therefore reads (the first two rows having been omitted):

$$R = [ \begin{matrix} & & \\ & & \\ - cos ( θ ) sin ( ϕ ) & sin ( θ ) & cos ( θ ) cos ( ϕ )\end{matrix} ]$$

The rotation matrix R can also be expressed as a set of orthogonal basis vectors i′, j′, k′ with each column expressing these vectors. Where orientation sensor 24 comprises an inclinometer, sensor 24 measures the gravitational force applied along the direction of each basis vector which is equivalent to obtaining the dot product of each of the basis vectors with a vertical vector k which is the direction of gravity:

a=i′·k

b=j′·k

c=k′·k

These values represent the bottom row of rotational matrix R (again the first two rows have been omitted):

$$R = [ \begin{matrix} & & \\ & & \\ a & b & c\end{matrix} ]$$

Therefore, the angles θ and γ can be determined from the following system of equations:

a=−cos(θ)sin(φ)

b=sin(θ)

c=cos(θ)cos(φ)

These equations resolve to:

$$θ = {sin}^{- 1} ( b )$$

$$ϕ = - {tan}^{- 1} ( \frac{a}{c} )$$

$$γ = 0$$

ECU 32 can therefore determine the full rotation matrix R responsive to information obtained from orientation sensor 24.

Referring again to FIG. 3, the method may continue with the step 50 of receiving a command through user interface 16 that is indicative of the desired movement of device 12 on display 30. The image generated on display 30 by imaging system 22 include a region of interest in body 14 and device 12. Using user interface 16, and with reference to the image on display 30, the clinician can enter an input or command to move device 12 from its current position as indicated in the image on display 30 to a new position relative to the region of interest in body 14 shown in the image.

The method may continue with the step 52 of generating a control signal to control movement of device 12 responsive to the command entered through user interface 16 and the viewing angle determined in step 42. The viewing angle as reflected in the image on display 30 establishing a relationship between the clinician/interface and the device 12 may not correlate with the relative positions or relationship of the clinician/interface 16 and the actual device 12 in body 14 because the various components of system 10 operate in different coordinate systems. In order to provide intuitive control to the clinician (i.e. such that an input or command entered through interface 16 to move the displayed device 12 in a certain direction relative to the region of interest of body 14 shown in display 30 results in the same movement of actual device 12 within body 14), ECU 32 must first register the various coordinate systems to one another. The coordinate systems of interface 16 and display 30 (as well as display 28) may be aligned such that a, for example, leftward movement of an input device in interface 16 would cause a corresponding leftward movement of an object on display 30 (and display 28). By virtue of having determined the viewing angle of imaging system 22 (and therefore the display angle of the image shown on display 30), ECU 32 can then relate the command entered through user interface 16 through appropriate translation and rotation in to a corresponding movement of device 12 within body 14 by, for example, applying the rotation matrix R to the commanded input. Further information regarding application of the matrix may be found in U.S. Published Patent Application No. 2010-0256558, the entire disclosure of which is incorporated herein by reference.

In accordance with one embodiment of the invention, the method may further include several steps intended to synchronize the viewing angle of the imaging system 22 with the display angle of a model generated by navigation system 20 on display 28. The method may therefore continue with the step 54 of determining a position of device 12 within body 14 and the step 56 of generating an image on display 28. The image generated on display 28 may include a model (e.g., a three-dimensional model) of a region of interest in body 14 and a representation of device 12 located relative to the model based on the determined position of device 12. Steps 54, 56 may be performed in a conventional manner using navigation system 20.

The method may continue with the step 58 of adjusting a display angle of the model responsive to the viewing angle of imaging system 22. In particular, ECU 32 may apply the rotational matrix R determined hereinabove to the individual coordinates of the model to adjust the display angle of the model and synchronize the imaging system viewing angle and the model display angle. Further explanation of the process for applying the matrix may again be found in U.S. Published Patent Application No. 2010-0256558, the entire disclosure of which is incorporated herein by reference.

A system and method in accordance with the present invention are advantageous because the system and method provide real time intuitive control of device 12. In particular, imaging system 22 provides a substantially real time display of the position of device 12 within body 14 thereby eliminating any lag in time between actual and displayed movements. The inventive system and method also provide a safeguard against the possibility that navigation system 20 could be rendered inoperable and leave the clinician without the ability to accurately control further movement of device 12.

Although several embodiments of this invention have been described above with a certain degree of particularity, those skilled in the art could make numerous alterations to the disclosed embodiments without departing from the scope of this invention. All directional references (e.g., upper, lower, upward, downward, left, right, leftward, rightward, top, bottom, above, below, vertical, horizontal, clockwise and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not as limiting. Changes in detail or structure may be made without departing from the invention as defined in the appended claims.

What is claimed is:

1. A system for synchronizing movement of a device within a body with a desired movement of said device commanded through a user interface, comprising:
an electronic control unit configured to:
determine a viewing angle of an imaging system configured to capture an image of said device within said body and to generate said image on a first display;
receive a command through said user interface, said command indicative of said desired movement of said device on said first display; and,
generate a control signal to control movement of said device within said body responsive to said command and said viewing angle.

2. The system of claim 1 wherein said electronic control unit is further configured to:
determine a position of said device within said body;
generate an image on a second display, said image including a model of a region of interest in said body and a representation of said device located relative to said model based on said position; and,
adjust a display angle of said model responsive to said viewing angle of said imaging system.

3. The system of claim 1 further comprising an orientation sensor connected to said imaging system, said orientation sensor generating an output signal indicative of said viewing angle of said imaging system.

4. The system of claim 3 wherein said orientation sensor comprises an inclinometer.

5. The system of claim 3 wherein said orientation sensor comprises a gyroscope.

6. The system of claim 3 wherein said orientation sensor comprises a magnetic field position sensor.

7. The system of claim 1 further comprising an orientation sensor disposed on said body, said orientation sensor comprising a magnetic field position sensor.

8. The system of claim 1 further comprising an orientation sensor disposed on a table supporting said body, said orientation sensor comprising a magnetic field position sensor.

9. The system of claim 1 wherein said electronic control unit is configured to obtain information from said image indicative of said viewing angle of said imaging system.

10. The system of claim 9 wherein said image is stored in a picture archiving and communications system and said electronic control unit is configured to access said image in said picture archiving and communications system.

11. The system of claim 9 wherein said image complies with the Digital Imaging and Communications in Medicine (DICOM) standard.

12. The system of claim 1 wherein said electronic control unit is configured to receive a signal generated by said imaging system indicative of said viewing angle of said imaging system.

13. The system of claim 1 further comprising said imaging system and wherein said imaging system comprises a fluoroscopic imaging system.

14. A method for synchronizing movement of a device within a body with a desired movement of said device commanded through a user interface, comprising the steps of:
determining a viewing angle of an imaging system configured to capture an image of said device within said body and to generate said image on a first display;
receiving a command through said user interface, said command indicative of said desired movement of said device on said first display; and,
generating a control signal to control movement of said device responsive to said command and said viewing angle.

15. The method of claim 14 further comprising the steps of:
determining a position of said device within said body;
generating an image on a second display, said image including a model of a region of interest in said body and a representation of said device located relative to said model based on said position; and,
adjusting a display angle of said model responsive to said viewing angle of said imaging system.

16. The method of claim 14 wherein said determining step includes the substep of receiving an output signal generated by an orientation sensor mounted on said imaging system.

17. The method of claim 16 wherein said orientation sensor comprises an inclinometer.

18. The method of claim 16 wherein said orientation sensor comprises a gyroscope.

19. The method of claim 16 wherein said orientation sensor comprises a magnetic field position sensor.

20. The method of claim 14 wherein said determining step includes the substep of obtaining information from said image indicative of said viewing angle of said imaging system.

21. The method of claim 14 wherein said determining step includes the substep of receiving a signal generated by said imaging system indicative of said viewing angle of said imaging system.

22. The method of claim 14 wherein said imaging system comprises a fluoroscopic imaging system.
